(19) United States
(12) Patent Application Publication
(10) Pub. No.: US 2018/0353775 A1
(43) Pub. Date: Dec. 13, 2018

(54) METHOD OF EVALUATING A DOSE AS FUNCTION OF DEPTH FOR NONUNIFORM X-RAY BEAMS

(71) Applicant: CONVERGENT R.N.R LTD., Tirat Caramel (IL)

(72) Inventors: Aharon BAR-DAVID, Nesher (IL); Zeev BURSHTEIN, Ness Tziona (IL); Avigail KELLER, Haifa (IL); Zeev HAREL, Kfar Saba (IL)

(21) Appl. No.: 16/104,578

(22) Filed: Aug. 17, 2018

Related U.S. Application Data
(63) Continuation of application No. PCT/IL2017/050199, filed on Feb. 15, 2017.
(60) Provisional application No. 62/296,610, filed on Feb. 18, 2016.

(51) Int. Cl. A61N 5/10; A61B 6/00

(52) U.S. Cl. CPC A61N 5/1071; A61N 5/1075; A61B 6/542

(57) ABSTRACT

A method of evaluating a maximal dose deposited by an X-ray beam within a medium comprises the steps of: (a) irradiating said medium by said X-rays beam penetrating into a depth of said medium along an axis of said X-ray beam; (b) measuring a dose distribution along said axis. The step of measuring said dose distribution further comprises measuring cross-sectional distributions with a predetermined incremental shift thereof along said beam axis, and calculating a 1-dimensional function of said maximal dose in dependence on said depth taken from maximum dose at each cross-sectional distribution at each depth.

### FIELD OF THE INVENTION

The use of this measure is related to all uses of X-ray producing device. Examples are: Radiotherapy, radiosurgery, imaging device using X-rays etc'. The main fields of use are medical, however one might find the use of the standard also in industrial filed such as Non-Destructive Tests (NDT) as well.

### BACKGROUND OF THE INVENTION

Percentage Depth Dose (PDD) curves have been used all over the world for many years in all the fields that use X-Ray radiation. This measure is intended to show the dose distribution as function of depth induced in the bulk of material by the penetration of an X-ray beam without going into the 3D isodose details of a beam penetrating the material in question.

Ordinary PDD curves are shown in a single shot in a single direction where the dose is measured along the center of the beam as function of depth inside the bulk of material, usually water (whose attenuation factor is similar to the one of tissues) or any other tissue equivalent phantom.

The purpose of showing the known PDD curves is usually to evaluate the largest dose absorbed by tissues as function of depth from an instrument having a specific X-ray spectrum when aimed at the material in question.

All today's devices (e.g. LINAC) produce beams whose cross-section has a smooth almost uniform transversal dose distribution with a (usually shallow) maximum at the center of the beam cross-section. The evaluation of largest dose at each depth is why today's PDDs are defined and measured along the center of the beam in a straight line where the maximum dose occurs on a transverse cross-section of the beam at each depth.

A question may arise when the beam irradiated is not uniform and/or not symmetrical, and/or the maximum is not at the center of the cross-section, additionally its points of maximum do not lie in a straight line along the beam propagation axis and may be not continuous.

To illustrate the problem clearly we take the example of a beam whose structure is hollow, i.e. zero radiation and therefore zero dose at the center of its cross-section, the whole shape of the beam may vary, e.g. like cone shape. The internal distributions of dose on cross-sections area at various depths may be at different points relative to the beams center at each depth. Thus, to show the dose inflicted by the instrument one cannot move in the empty middle of the beam. Additionally if the internal structure of the beam has a shape where its maximum dose points occur on various different distances from the beam axis that change arbitrarily, or may jump at different places or angles rotationally around the beams axis they may be located not along a straight line and/or not in a continuous line.

In the case of non-uniform medium in which the X-ray beam propagates, PDD characterization does not provide adequate information about dose distribution deposed in the medium. There is a long-felt and unmet need to provide dose characterization as a function of depth indicating the maximal dose in every depth along the penetration of the X-ray beam.

### SUMMARY OF THE INVENTION

It is hence one object of the invention to disclose method of evaluating a maximal dose deposited by an X-ray beam within a medium. The aforesaid method comprises the steps of: (a) irradiating said medium by said X-rays beam penetrating into a depth of said medium along an axis of said X-ray beam; and measuring a dose distribution along said axis.

It is a core feature of the present invention to provide a step of measuring the dose distribution further comprising measuring cross-sectional distributions with a predetermined incremental shift thereof along said axis, determining a maximal dose in each cross-sectional distribution and calculating a maximum of said deposited dose as a function of depth. The Maximal dose may appear along a 3D curve that might be not continuous.

Another object of this disclosure is to disclose the step of measuring cross-sectional dose distribution comprising normalizing obtained data of measurements to said maximal dose therewithin.

### BRIEF DESCRIPTION OF THE DRAWINGS

In order to understand the invention and to see how it may be implemented in practice, a plurality of embodiments is adapted to now be described, by way of non-limiting example only, with reference to the accompanying drawings, in which:

FIG. 1 is a schematic illustration of a common LINAC beam.

FIG. 2 shows the color description of the dose intensity used in all figures at items showing beam structure where dose intensity is illustrated.

FIG. 3 a-d show examples of beam structures inside material depositing non-homogenous dose distribution.

FIG. 4a shows an example of longitudinal cut through material showing a cone shaped beam depositing non-homogenous dose with non-homogenous ring like shaped regions of dose distribution with its path through maximum dose points at each depth. FIG. 4b shows the Maximal Percentage Depth Dose (MPDD) along the same depth axis as FIG. 4a.

FIG. 5a shows an example of longitudinal cut through material showing a complex shape which converges to a depth and diverges beyond that depth deposing non homogenous dose distribution with its path through maximum dose points at each depth. FIG. 5b shows the Maximal Percentage Depth Dose (MPDD) along the same depth axis as FIG. 5a.

FIG. 6a shows an example of a cone shaped X-ray beam penetrating material with internal intensity structure of rings. FIG. 6b shows an example of two-dimensional measuring device located at specific depth measuring the local depth dose distribution from which the maximum dose can be obtained.

### DETAILED DESCRIPTION OF THE INVENTION

The following description is provided, alongside all chapters of the present invention, so as to enable any person skilled in the art to make use of the said invention, and sets forth the best modes contemplated by the inventor of carrying out this invention. Various modifications, however, are adapted to remain apparent to those skilled in the art, since the generic principles of the present invention have been defined specifically to provide a method for the a measure of evaluation of the action of X-ray beams especially in cases where the beam has at least one of the situations where it is not uniform, not symmetrical, not continuous on the transverse direction and or goes through changes along the propagation axis—some or all of them together.

The present invention solves problems regarding the following issues:

Percentage Depth Dose (PDD) refers to the dose which is deposited by todays existing device e.g. LINAC, at the center of the beam at each depth. Such beams have a cross-section where a maximum dose appears at the center of this cross-section. The maximum dose registration is necessary to make sure that no higher dose exists anywhere on the cross-section at each depth. Thus, for a non-uniform beam, this type of measurements is not adequate in order to achieve the aforesaid purpose, due to the fact that the maximum does not appear generally in the center.

Furthermore, when the function of the maximum dose or maximum dose percentage deposited depending of penetration depth is obtained, it should be taken into account that not all maximum points may lie on the center or the axis of the X-ray beam. It is different from the known PDD because it provides the maximal points at each depth regardless their transversal location. It should be emphasized that, in the case of a non-uniform beam, the position of maximal deposited dose can deviate from the beam axis, and might be non-continuous.

The term “Maximal Dose Depth (MDD)” refers to a one-dimensional function of depth that shows the maximum dose at each depth. The units of the function are units of real dose, e.g. Gray. The depth axis is continuous only along the depth direction; thus the location of the points of maxima may lie on a 3 dimensional curve that may be nonlinear and/or not continuous.

The term “Maximal Percentage Dose Depth (MPDD)” is similar to MDD expressed in relative units (per cents). The percentage is relative to the maximum dose of all maxima at all depth, i.e. the maximum of the corresponding MDD function. The difference of the MPDD from the known PDD is that a PDD takes the dose as function of depth along a straight line and is defined at the center of the beam, which in a non-uniform complex beam may not lie at the maximum of the beam, whereas MPDD is specifically related to the maxima at each depth, thus it may take the highest value of dose at each depth going through a possibly 3 dimensional curve, which might be not along a straight line and might be not continuous, i.e. going through non continuous points on some transversal plane at some depths although covering all points on the propagation axis.

The term “envelope’ refers to an overall shape of the beam limited by the criterion of the dose of 1% of its maximum on the local cross-section plane at each depth. The purpose is to relate to beams whose internal structure may differ from its outer structure. For example a beam that has an overall shape of a cone might have internal structure where the points of maximum dose don't lie on a cone shape at all (changing arbitrarily).

The term “TCS” refers to the beam's local 2 dimensional Transversal Cross-Sectional plane (relative to the beam propagation direction) at each depth point.

The term “LCS” refers to Longitudinal Cross-Section cut along the beam propagation direction.

Example of use of the last 2 terms can be: TCS dose distribution—to say transversal dose distribution.

Another example: LCS dose distribution is a longitudinal cut showing dose distribution along the beam propagation.

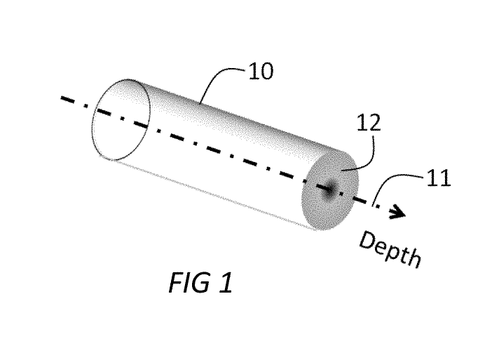

Reference is now made to FIG. 1 illustrating a schematic shape of a beam provided by a linear particle accelerator (LINAC). The envelope of the beam (10) in this case is cylindrical. The propagation axis (11) is in the direction of depth inside the material that the X-ray penetrates.

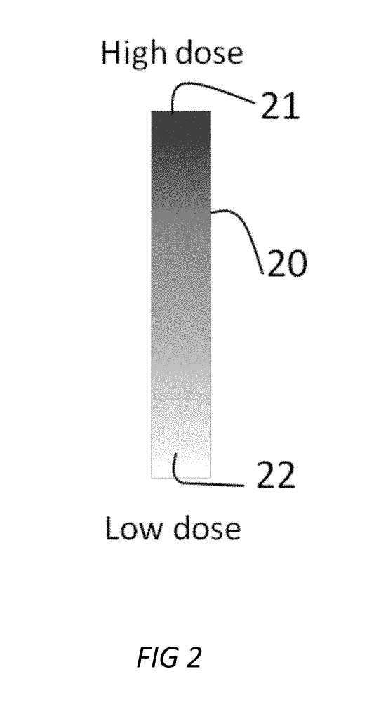

Numeral 12 refers to a cross-sectional dose distribution. The color code corresponding to different doses is shown in FIG. 2. As can be seen the dose is almost uniform and has a maximum in the central portion.

Reference is now made to FIG. 2 illustrating the dose distribution value color code in all relevant parts of all figures by means of a color bar (20). The lower edge (22), white, relates to low dose and the upper end of the bar (21), dark gray color, relates to higher dose.

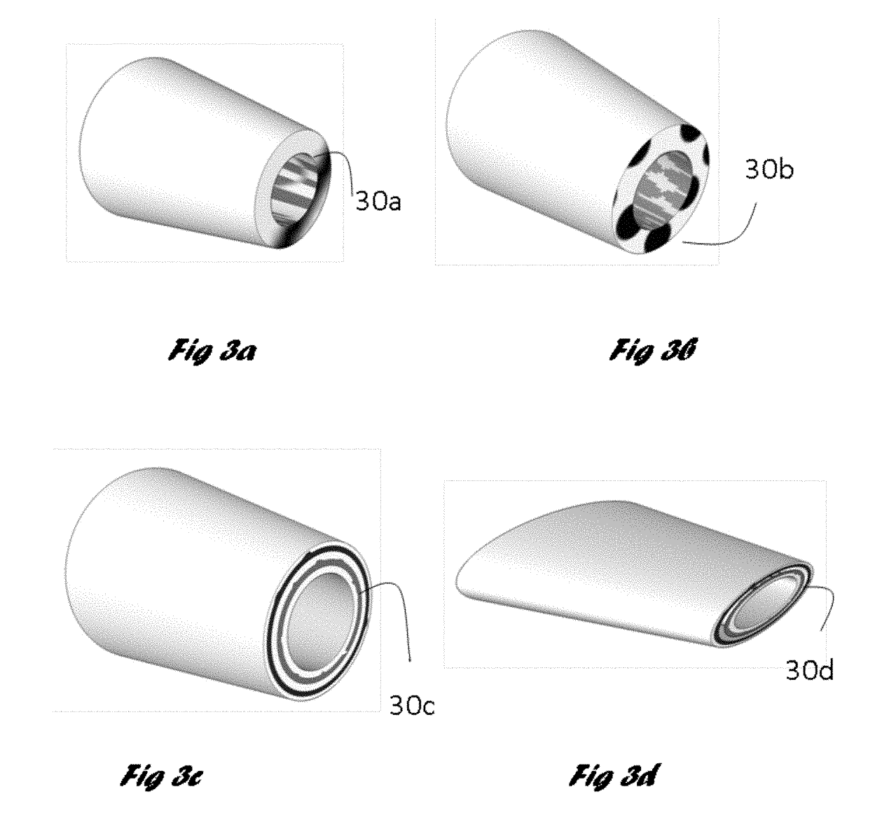

Reference is now made to FIG. 3 showing examples of X-ray beams having a cone shaped envelope with a hollow central part. The TCS dose distributions are shown at the front of each FIG. 30a)-(30d). The color distribution on them should be taken as the color code from FIG. 2. For example (30a) and (30b) show completely non-symmetrical TCS dose distributions. The TCS dose distribution (30b) shows several blobs of high dose not at the center. The TCS dose distribution on (30c) is of several relative thin ring areas of high dose. Since this example has a cone shaped envelope, one can envision that the TCS high dose rings resemble thin cones of high dose in 3 dimensions. FIG. 3d shows a different envelope of an example of elliptical cone.

In general the beam's envelope of arbitrary shape and structure is in the scope of the present invention.

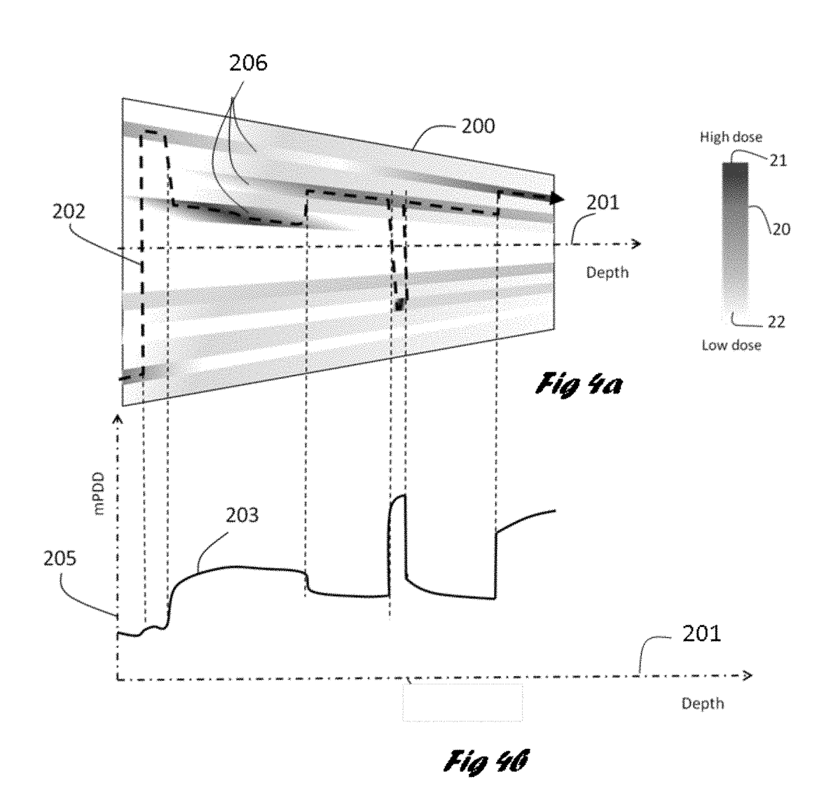

Reference is now made to FIG. 4a Showing an example of an LCS dose distribution of a cone envelope (200) with internal thin cons like dose regions (206) (which would appear as rings on TCS dose distributions). The regions are not uniform and not symmetric. Thus, the track (202) that follows this MPDD (203) along the depth axis (201) is not along a straight line but rather on a line with broken and curved parts that go through the highest dose regions (the darkest areas of each depths in the figure).

Reference is now made to FIG. 4b presenting MPDD curve (203) which shows the dose value at each depth along the depth axis (201). Dose value is on the Y axis (205). FIG. 4b shows the correspondence between dependences of the LCS on dose distribution and its MPDD (see dashed lines) on the penetration depth.

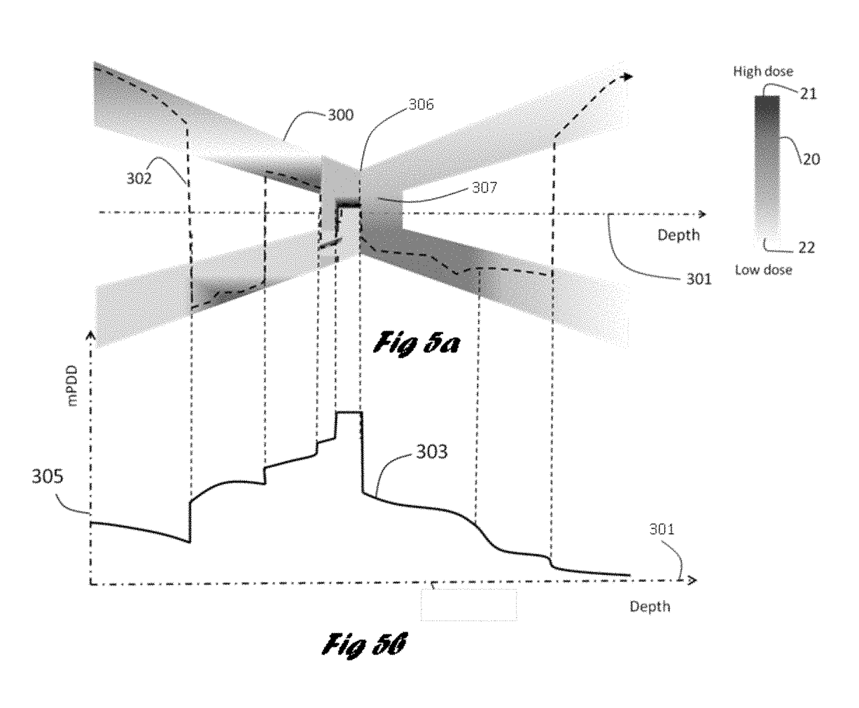

Reference is now made to FIG. 5a presenting an example of a more complex beam. Its envelope (300) has a hollow cone structure at shallow depths and converges to a certain depth (306). Beyond this depth it diverges again to a hollow reversed cone. In the middle part (307) it's not hollow and fills the whole bulk. The dose distribution in this example is arbitrary. The MPDD track (302) is again a nonlinear broken curved line that goes through the maximum dose at each depth (darkest areas at each depth).

Reference is now made to FIG. 5b. The MPDD (303) related to the beam in FIG. 5a is drawn again to match the axis of depth (301) (see dashed lines).

The examples of FIGS. 4 and 5 show, for graphical convenience, an LCS dose analysis on a plane longitudinal cut, however, the curve along which the MPDD is taken does not have to lie at all on a plane. The curve may lie on a 3 dimensional arbitrarily swiveled curved track that may also be not continuous.

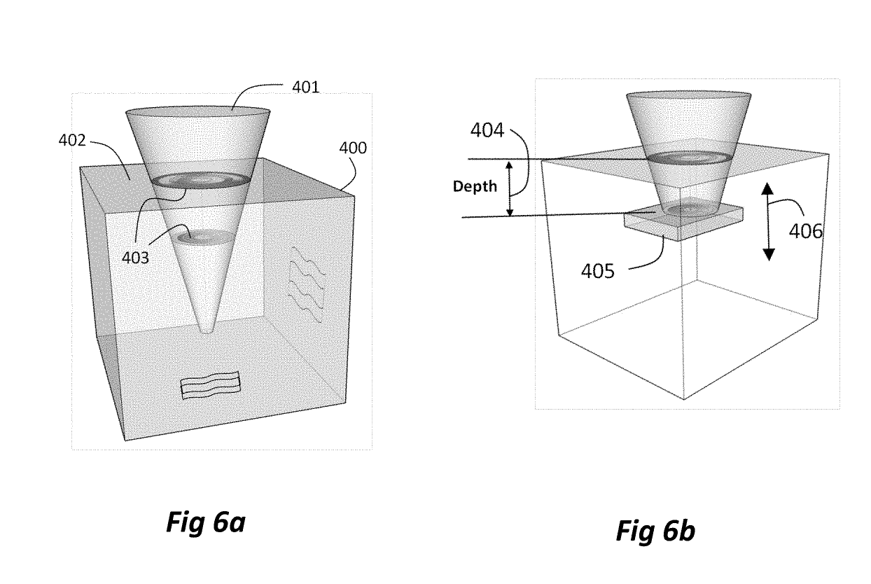

Reference is now made to FIG. 6a which shows obtaining the non-uniform properties and changing its shape along the propagation axis. FIG. 6a shows an example of a cone shaped X-ray beam (401) penetrating a bulk of material (400) at surface of interface (402) with example of internal intensity distribution of rings (403). A method of measuring MDD or MPDD is illustrated in FIG. 6b. A 2-dimensional detecting device (405) is placeable at each depth (404) such that a 2-dimensional transversal distribution is taken. The detecting device is movable along the beam propagation direction with a predetermined increment (406), and maximal dose is obtained at each measurement.

1.-2. (canceled)

3. A device for evaluating a maximal dose deposited by a non-uniform X-ray beam within a uniform medium; said device comprising:
a. an source of said non-uniform X-ray beam irradiating said medium; said X-rays beam penetrating into a depth of said uniform medium along an axis of said X-ray beam;
b. a 2-dimensional detecting device displaceable along said axis;
wherein said 2-dimensional detecting device is configured for measuring a transversal cross-sectional dose distribution provided by said non-uniform X-ray beam at a plurality of successive depths along said axis with a predetermined incremental depth shift such that an 1-Dimensional function of a maximal measured dose over each transversal cross-sectional distribution of said non-uniform X-ray beam in dependence on said depth is provided.

4. The device of claim 2, wherein said cross-sectional dose distribution is normalized to said maximal dose therewithin.
